SEQUENCE LISTING (7 sequences)

SEQ ID NO: 1 - protein, 41 aa, Homo sapiens
QQNVNGAFAELRKLIPTHPPDKKLSKNEILRLAMKYINFLA

SEQ ID NO: 2 - protein, 28 aa, Homo sapiens
VRRIFTNSRERWRQQNVNGAFAELRKLI

SEQ ID NO: 3 - protein, 26 aa, Homo sapiens
PTHPPDKKLSKNEILRLAMKYINFLA

SEQ ID NO: 4-5 - skipped (2 entries)

SEQ ID NO: 6 - protein, 17 aa, Artificial Sequence
  Description of Artificial Sequence: Synthetic Peptide
AWSFRVSYRGISYRRSR

SEQ ID NO: 7 - protein, 10 aa, Artificial Sequence
  Description of Artificial Sequence: Synthetic Peptide
RRLSYSRRRF

(19) United States
(12) Patent Application Publication
(10) Pub. No.: US 2007/0161553 A1
(43) Pub. Date: Jul. 12, 2007

(54) Angiogenesis inhibitors, compositions containing same and use thereof for treating diseases related to angiogenetic deregulation

(75) Inventors: Daniele Mathieu, Montferrier-Sur-Lez (FR); Jamal Temsamani, Nimes (FR); Michel Kaczorek, Montferrier-Sur-Lez (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: SYNT:EM, NIMES (FR)

(21) Appl. No.: 10/587,874

(22) PCT Filed: Feb. 2, 2005
(86) PCT No.: PCT/FR05/00222

§ 371 Date: Nov. 16, 2006

(30) Foreign Application Priority Data
Feb. 2, 2004 (FR) 0400964
Jun. 2, 2004 (FR) 0405954

(51) Int. Cl. A61K 38/55; C07K 14/47

(52) U.S. Cl. 514/12; 514/13; 530/324; 530/326

(57) ABSTRACT

A peptide molecule that interferes with an HLH domain of TAL-1 including at least 10 successive amino acids from the HLH domain of TAL-1 of sequence: QQNVNGAFAELRKLIPTHPPDKKLSKNEILRLAMKYINFLA (SEQ ID No. 1) or an equivalent sequence; and a pharmaceutical composition comprising at least one peptide molecule as an active ingredient, and an acceptable vehicle.

### RELATED APPLICATION

This is a §371 of International Application No. PCT/FR2005/000222, with an international filing date of Feb. 2, 2005 (WO 2005/075509 A1, published Aug. 18, 2005), which is based on French Patent Application Nos. 04/00964, filed Feb. 2, 2004, and 04/05954, filed Jun. 2, 2004.

### TECHNICAL FIELD

This disclosure relates to treatment of diseases related to an angiogenesis disorder such as cancers, arteriosclerosis and diabetes. The disclosure relates to specific angiogenesis inhibitors able to interfere with the different interactions of TAL-1 likely to be conjugated with vectors such as peptides, particles (liposomes, nanoparticles, etc.) and polymers. The disclosure further relates pharmaceutical compositions containing such inhibitors and their use in the treatment of diseases related to angiogenesis, more specifically in the treatment of cancers, arteriosclerosis and diabetes.

### BACKGROUND

Angiogenesis, process of remodelling pre-existing capillaries, leads to the formation of new vessels (Riseau, Nature 386; 671 (1977)). In physiological conditions, angiogenesis is closely regulated under the control of a local balance between stimulator (angiogenics) and inhibitor (angiostatics) factors. In the adult, most of the cells of the endothelium (over 99%) are in the quiescent state. In response to an angiogenic signal, the endothelial cells (ECs) come out of their dormancy and become “activated” to trigger a programme of angiogenesis. These complex processes include the movement of ECs to leave the capillaries and their migration to the angiogenic site, their proliferation and their differentiation to form new capillaries.

An angiogenesis disorder is observed in certain diseases such as arteriosclerosis, diabetes and tumours (Carmeliet, Nat Med 6; 389 (2000)). When the tumours reach a critical size creating conditions of hypoxia, the tumour cells give off cytokines, such as VEGF or bFGF, known to initiate or stimulate angiogenesis. Angiogenesis, required for the growth of tumours, is also a main factor in the propagation of malignant cells in the organism (metastases).

In spite of undeniable progress in the fight against certain cancers, it is necessary to recognize that cancers are progressing globally and that they are a major cause of death. The discovery of new drugs is one of the priorities of medical research. Recent progress in scientific knowledge on tumoral angiogenesis indicates that a treatment based on anti-angiogenesis may provide a new way to control the growth of tumours in cancer patients.

Several anti-angiogenetic strategies have been developed to inhibit the function of the activator factors (VEGF or bFGF), in particular with neutralizing antibodies directed against the cytokine or against its receptor. Other anti-angiogenetic molecules have been derived from the physiological inhibitors present in the haemostatic system, such as plasminogen (angiostatin), collagen (endostatin) or even fibrinogen (alphastatin). These molecules inhibit the adhesion of the ECs to the extracellular matrix required for their morphogenesis.

Another strategy that has been very little explored to date is to directly target the intracellular events of the endothelial cell in response to angiogenetic stimuli. Therefore, the applicant has demonstrated that TAL-1, a transcription factor from the basic-Helix-Loop-Helix (bHLH) family, specifically modulates the angiogenetic response of the endothelial cells. It controls their migration properties and stimulates their differentiation into capillaries (Lazrak et al., J Cell Sci in press (2004)). Homologous recombination experiments in the mouse have demonstrated that the tal-1 gene is required for certain stages in haematopoietic and vascular development. Tal-1−/− embryos (not expressing tal-1) present a defective angiogenesis in the yolk sac that seems to reflect an intrinsic defect in the endothelial cells (Visvader et al., Genes Dev 12; 473 (1998)). In the adult, except of certain haematopoietic progenitors of the bone marrow, TAL-1 protein is expressed in the small vessels in formation, but not in the quiescent mature endothelium (Kallianpur et al., Blood 83; 1200 (1994)). Significantly, a high level of expression of TAL-1 is observed in the vasculature of human tumours (Chetty et al., J Pathol 181; 311 (1997)). TAL-1 is therefore a marker of the angiogenetic ECs. Therefore, we targeted TAL-1 to inhibit angiogenesis.

Molecules able to specifically block the activity of TAL-1 in endothelial cells have been developed. The HLH motif of TAL-1 is required for all of the activities of the TAL-1 factor known to date: fixation on DNA or interaction with other nuclear factors, in particular E47 or LMO2 (Hsu et al., Proc. Natl. Acad. Sci. 91; 3181 (1994); Vitelli et al., Mol Cell Biol 20; 5330 (2000)) (FIG. 1). Peptide inhibitors able to enter into competition with the HLH domain of TAL-1 have also been developed. These peptide inhibitors were then coupled with peptide vectors to enable and/or improve their internalization in the cells. That work is described in FR 97/10297, filed on 8 Dec. 1997.

### SUMMARY

This invention relates to a peptide molecule that interferes with an HLH domain of TAL-1 including at least 10 successive amino acids from the HLH domain of TAL-1 of sequence: QQNVNGAFAELRKLIPTHPPDKKLSKNEILRLAMKYINFLA (SEQ ID No. 1) or an equivalent sequence.

This invention also relates to a pharmaceutical composition including at least one peptide molecule as an active ingredient and an acceptable vehicle.

This invention further relates to a method of preventing and/or treating diseases related to angiogenesis, including administering a therapeutically effective amount of the pharmaceutical composition.

This invention still further relates to a peptide molecule including a sequence selected from the group consisting of QQNVNGAFAELRKLIPTHPPDKKLSKNEILRLAMKYINFLA (SEQ ID No. 1), VRRIFTNSRERWRQQNVNGAFAELRKLI (SEQ ID No. 2), PTHPPDKKLSKNEILRLAMKYINFLA (SEQ ID No. 3) and a sequence equivalent to the sequences.

This invention further still relates to a pharmaceutical composition including at least one peptide molecule as an active ingredient associated with a vector.

This invention yet again relates to a method of preventing and/or treating diseases related to angiogenesis, including administering a therapeutically effective amount of the pharmaceutical composition associated with a vector.

### BRIEF DESCRIPTION OF DRAWINGS

Other advantages and characteristics of the invention will appear upon reading the following examples concerning the preparation of a compound consisting of a TAL-1 inhibitor and a peptide vector as well as on their in vitro and in vivo effects. Reference will be made to the drawings:

FIG. 1 represents the dimmer HLH;

FIG. 2 illustrates the in vitro inhibition of the interaction of TAL-1 and E47 by the different inhibitors;

FIG. 3 illustrates the inhibition of the in vitro inhibition of the interaction of TAL-1 and E47 by the inhibitor coupled with a peptide vector;

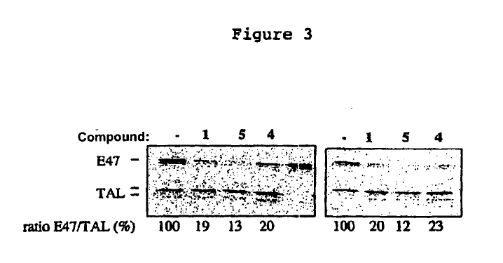

FIG. 4 illustrates the effect of compounds on the survival of HUVEC endothelial cells;

FIG. 5 illustrates the in vitro effect of one of the compounds in the invention on tubulogenesis of human endothelial cells after 22 hours of culture;

FIG. 6 presents the in vivo effect of the compounds in the invention on angiogenesis in the mouse by macroscopic analysis of Matrigel implants whether or not complemented with the compounds in the invention; and

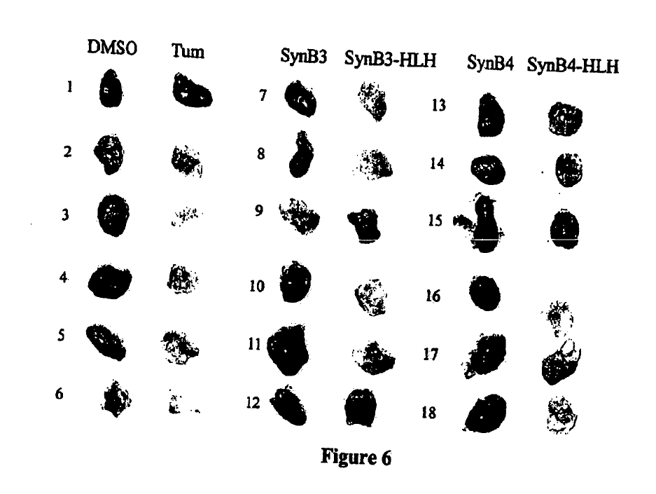

FIG. 7 presents the in vivo effect of the compounds in the invention on angiogenesis in the mouse by assay of the haemoglobin contained in the said Matrigel implants.

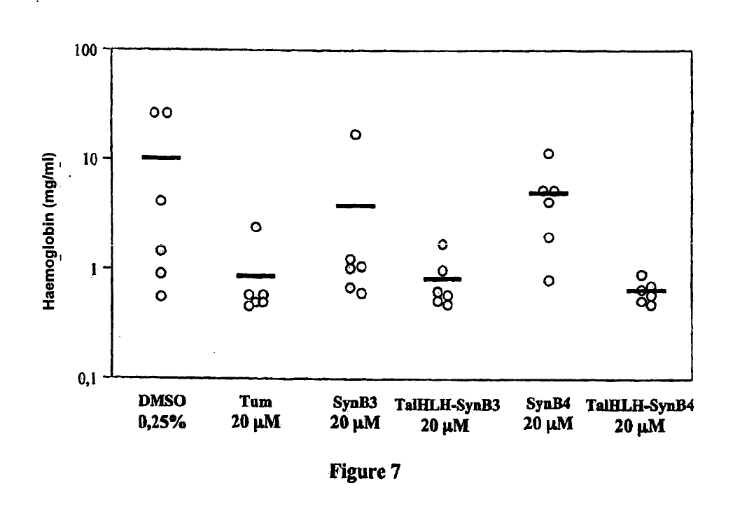

### DETAILED DESCRIPTION

In the peptide sequences presented below, the amino acids are represented by their code with one letter, but they may also be represented by their code with three letters according to the following nomenclature:

| | | |
|---|---|---|
| A | Ala | alanine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | valine |
| W | Trp | tryptophane |
| Y | Tyr | tyrosine |

We provide a peptide inhibitor (also called “peptide molecule”) able to interfere with the HLH domain of TAL-1, consisting of or comprising at least 10 successive amino acids and, preferably, at least 15 successive amino acids from the HLH domain of TAL-1 of sequence:
QQNVNGAFAELRKLIPTHPPDKKLSKNEILRLAMKYINFLA (SEQ ID No. 1)
   or an equivalent sequence.

“Equivalent sequence” refers to the sequence of a known variant of the HLH domain of TAL-1 as well as a sequence having one or several substitutions, deletions and/or additions of amino acids with respect to the sequence SEQ ID No. 1, the substitutions, deletions and/or additions of amino acids not modifying the activities of the TAL-1 factor thereby obtained as its fixation on DNA or its interaction with other nuclear factors, in particular E47 or LMO 2. Those skilled in the art are aware of different techniques to verify the activity of the TAL-1 transcription factor thereby obtained. In addition, “equivalent sequence” refers to peptides having a post-translational modification and/or a chemical modification, in particular a glycosylation, an amidation, an acylation, an acetylation, a methylation as well as the peptides that carry a protective group. The derivatives of the peptides may also be those where one or several amino acids are enantiomers, diastereoisomers, natural amino acids of conformation D, rare amino acids in particular hydroxyproline, hydroxylysine, allo-hydroxylysine, 6-N-methylysine, N-ethylglycine, N-methylglycine, N-ethylasparagine, allo-isoleucine, N-methylisoleucine, N-methylvaline, pyroglutamine, aminobutyric acid and the synthetic amino acids, in particular ornithine, norleucine, norvaline, cyclohexyl-alanine and the omega amino-acids. The derivatives also cover the retropeptides and the retroinversopeptides, as well as the peptides whose lateral chain of one or several amino acids is substituted by groups that do not modify the anti-microbial activity of the peptides in the invention.

“Peptide molecule” refers to a molecule consisting of or comprising at least one peptide sequence, the molecule may also include additional chemical entities other than amino acids.

Preferably, the inhibitor or peptide molecule is chosen from among compound 1 of sequence: QQNVNGAFAELRKLIPTHPPDKKLSKNEILRLAMKYINFLA (SEQ ID No. 1), compound 2 of sequence: VRRIFTNSRERWRQQNVNGAFAELRKLI (SEQ ID No. 2) and compound 3 of sequence: PTHPPDKKLSKNEILRLAMKYINFLA (SEQ ID No. 3) or a sequence equivalent to the sequences. Therefore, the inhibitor consists of or comprises a sequence chosen from among the following sequences:

(SEQ ID No. 1)

QQNVNGAFAELRKLIPTHPPDKKLSKNEILRLAMKYINFLA,

(SEQ ID No. 2)

VRRIFTNSRERWRQQNVNGAFAELRKLI
and

(SEQ ID No. 3)

PTHPPDKKLSKNEILRLAMKYINFLA

or a sequence equivalent to the sequences.

The inhibitor or peptide molecule may be associated with a vector. In an advantageous manner, this vector is able to increase the transport of the inhibitor in the cells and target organs.

Those skilled in the art are aware of different types of peptide vectors that may be used. Therefore, by way of example and in a non-limiting manner, the vector is chosen from among the group comprising:
a linear peptide derived from protegrins or tachyplesins (FR 97/10297),
a linear peptide comprising a domain of transduction such as the domains of transduction of Tat protein of HIV-1 (Fawell et al., Proc. Natl. Acad. Sci. 91; 664 (1994); Schwarze et al., Science 285; 1569 (1999)) and the domains of transduction derived from the third helix of Antennapaedia (Derossi et al., J. Biol. Chem 269, 10444 (1994); U.S. Pat. No. 5,888,762),
particles such as liposomes (Dass and Su. (2001) Drug Deliv. 8:191-213) or nanoparticles (Panyam and Labhasetwar (2003) Adv Drug Deliv Rev. 55:329-47; Douglas et al. (1987) Crit Rev Ther Drug Carrier Syst. 3:233-61),
polymers such as polyethylene glycol (PEG) (Greenwald et al. (2003) Adv Drug Deliv Rev. 55:217-50).

We provide a linear peptide derived from Protegrins, a peptide that complies with the following formula (I):
BX(X or B)BXXXXBBBXXXXXXB (I)
and as a linear peptide derived from Tachyplesin, a peptide that complies with the following formula (II):
(B or X)XXXBXXXBXXXXBBXB (II),
where:
the identical or different B groups represent an amino acid residue whose lateral chain bears an alkaline group, and
the identical or different X groups, represent an aliphatic or aromatic amino acid residue
   or a fragment of them consisting of a sequence of at least 5 and preferably at least 7 successive amino acids of peptides from formulae (I) or (II).

We also disclose the use of a vector as defined above to vectorize in the cells, tissues and/or organs, a peptide inhibitor as defined above.

The bond between the inhibitor or peptide molecule of Tal-1 able to interfere with the different interactions of TAL-1 as defined above and the vector is chosen from among a covalent bond, a hydrophobic bond, an ionic bond, a cleavable bond or a non cleavable bond in physiological media or inside cells.

This bond may be direct or indirect by means of a linker and carried out by means of a functional group naturally present or introduced either on the vector, or on the inhibitor, or on both. This linker, if present, should be acceptable considering the chemical nature and size of both the vector and inhibitor. By way of example and in a non-limiting manner, we can cite linkers containing alkyl, aryl, aralkyl or peptide groups, esters, aldehydes or alkyl, aryl or aralkyl acids, anhydride, sulfhydryl or carboxyl groups such as the derivatives of maleymil benzoic acid, maleymil propionic acid and succinimidyl derivatives, the derivatives of cyanogenic bromide or chloride, carbonyldiimidazole, succinimide esters or sulphonic halogenures.

As functional groups, it is possible to mention: —OH, —SH, —COOH, or —NH₂. Therefore, the inhibitor may be bound by covalent bonds at the level of the N-terminal or C-terminal ends or at the level of the lateral chains of the peptide vector.

One type of preferred bond between the angiogenesis inhibitor and the vector involves at least one disulphide bridge.

In a particularly advantageous manner, the inhibitor is chosen from among the following two compounds:

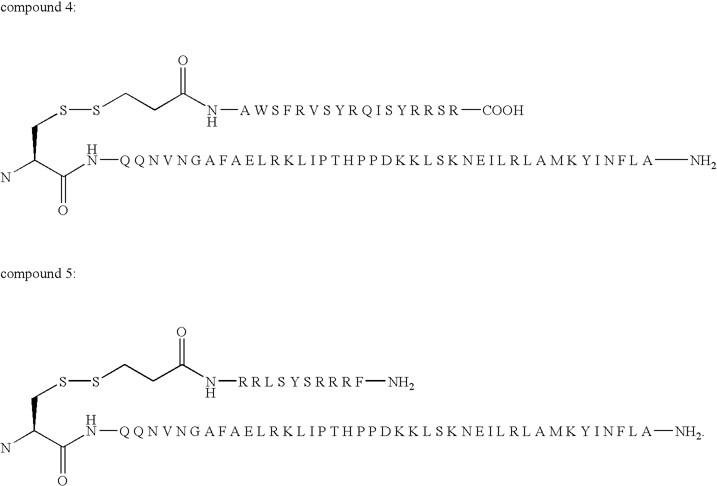

We provide pharmaceutical compositions comprising, as an active ingredient, at least one peptide inhibitor (or molecule) as defined above, advantageously associated in the composition with an acceptable vehicle.

The pharmaceutical composition may be in an appropriate form for parenteral, oral, rectal, nasal, transdermal, pulmonary or central administration. “Vehicle” refers to any substance that is added to the inhibitor of the invention to favour its transport, avoid substantial degradation in the composition and preserve its inhibiting properties. The vehicle is chosen as a function of the type of application listed above the composition.

We further provide a method for the treatment of diseases related to an angiogenesis disorder such as cancers, arteriosclerosis and diabetes, consisting of the administration, to a subject suffering from such a disease, of an efficient quantity of an inhibitor or a composition as described above.

We provide for the use of an inhibitor or a composition as described previously for the preparation of a drug intended to treat diseases related to angiogenesis, and more specifically diseases related to an angiogenesis disorder, preferably the treatment of cancers, arteriosclerosis and diabetes.

We provide for the use of a compound able to inhibit the interaction between the HLH domain of TAL-1 and its partner E47 for the preparation of a drug intended to prevent and/or treat diseases related to angiogenesis and, preferably, the treatment of cancers, arteriosclerosis and diabetes.

A compound able to inhibit the interaction between the HLH domain of TAL-1 and its partner E47 may be a competitive inhibitor of the HLH domain of TAL-1. Such a compound may be a peptide molecule as defined above.

A compound able to inhibit the interaction between the HLH domain of TAL-1 and its partner E47 may be a compound able to inhibit the fixation of TAL-1 on its partner E47. Such a compound may be an antibody recognising either an epitope at the level of the HLH domain of TAL-1, or an epitope at the level of the HLH domain of E47.

The applications and uses considered for the inhibitors (inhibitors or peptide molecules) previously described apply mutates mutandis to the compounds able to inhibit the interaction between the HLH domain of TAL-1 and its partner E47 (methods of treatment, pharmaceutical compositions, etc.).

Finally, we provide a method to identify a biologically active compound likely to be used in the prevention and/or treatment of diseases related to angiogenesis and, preferably, the treatment of cancers, arteriosclerosis and diabetes, consisting of detecting the inhibition of the interaction between the HLH domain of TAL-1 and its partner E47 in the presence of the compound.

By “biologically active compound”, we mean any natural or synthetic chemical compound such as, by way of example but in a non-exhaustive manner, proteins, polypeptides, peptides, aptamers, lipoproteins, polysaccharides, small molecules, non-peptide molecules, etc. After the identification of the biologically active compound, it will be easy to test its action in the prevention and/or treatment of diseases related to angiogenesis and, preferably, the treatment of cancers, arteriosclerosis and diabetes using methods familiar to one skilled in the art.

Those skilled in the art have different techniques available to verify whether the biologically active compound to test is able to inhibit the interaction between the HLH domain of TAL-1 and its partner E47.

One of the methods comprises the following stages:
(a) putting into contact the TAL-1 protein (or a fragment of this protein comprising the HLH domain), the transcription factor E47 (or a fragment of this factor comprising the domain that interacts with TAL-1) and the biologically active compound to test,
(b) immunoprecipitate either the TAL-1 protein (or a fragment of this protein containing the HLH domain), or transcription factor E47 (or a fragment of this factor comprising the domain that interacts with TAL-1),
(c) if, at stage (b), the TAL-1 protein (or a fragment of this protein containing the HLH domain), is immunoprecipitated, detect in the immunoprecipitate obtained in stage (b), the presence of transcription factor E47 (or a fragment of this factor comprising the domain that interacts with TAL-1),
(d) if, at stage (b), transcription factor E47 (or a fragment of this factor containing the domain that interacts with TAL-1) is immunoprecipitated, detect in the immunoprecipitate obtained in stage (b), the presence of the TAL-1 protein (or a fragment of this protein comprising the HLH domain),
   in case transcription factor E47 (or a fragment of this factor comprising the domain that interacts with TAL-1) is not present in stage (c) or if protein TAL-1 (or a fragment of this protein comprising the HLH domain) is not present in stage (d), the compound is an agent likely to be used in the prevention and/or treatment of diseases related to antiogeneisis and, preferably, the treatment of cancers, arteriosclerosis and diabetes.

Those skilled in the art have different proteins available that are likely to be used in the method. By way of example, protein E47 that is useable may be the protein from Gallus gallus present in Genbank under number CAE30454 or the protein from Mus musculus present in Genbank under number AAK18618. In the same way, the TAL-1 protein that may be used is the protein from Homo sapiens present in Genbank under number P17542 or the protein from Mus musculus present in Genbank under number P22091. In these sequences, one skilled in the art knows the domains of particular interest.

In stage (b) of the method, those skilled in the art know which type of antibody used as a function of the protein to immunoprecipitate (specific antibody of transcription factor E47 (or a fragment of this factor comprising the domain that interacts with TAL-1) or a specific antibody of the TAL-1 protein (or a fragment from this protein comprising the HLH domain)).

The detection, in stage (c), of the presence, in the immunoprecipitate obtained in stage (b), of transcription factor E47 (or a fragment of this factor comprising the domain that interacts with TAL-1) may be carried out with any known technique such as, for example, a Western Blot using a specific antibody for transcription factor E47 (or a fragment of this factor comprising the domain that interacts with TAL-1), an assay of the activity of transcription factor E47 (or a fragment of this factor comprising the domain that interacts with TAL-1) or a method using a labelled transcription factor E47 (or a fragment of this factor comprising the domain that interacts with TAL-1), the labelling possibly being radioactive labelling.

The implementation may be carried out mutates mutandis for the TAL-1 protein (or a fragment of this protein comprising the HLH domain) in stage (d).

The method may comprise the following stages:
(a′) putting into contact protein TAL-1 (or a fragment of this protein comprising the HLH domain), transcription factor E47 (or a fragment of this factor comprising the domain that interacts with TAL-1) and the biologically active compound to test,
(b′) make migrate on a non-denaturant polyacrylamide gel the mixture obtained in stage (a′),
(c′) visualize the absence or presence of TAL-1 complex (or a fragment of this protein comprising the HLH domain) and E47 (or a fragment of this factor comprising the domain that interacts with TAL-1).

The absence of this complex in stage (c′) reveals the inhibiting effect of the active compound tested and therefore this compound is an agent likely to be used in the prevention and/or treatment of diseases related to angiogenesis and, preferably, the treatment of cancers, arteriosclerosis and diabetes.

In stage (c′) of the method, different known techniques may be used. By way of example, it is possible to carry out a Western blot using specific antibodies from one of proteins TAL-1 or E47 or their fragments.

I—Chemical Synthesis

1) Synthesis of Inhibitors of Free and Vectorized TAL-1

The peptides were assembled on a solid phase according to a Foc/tu strategy, cleaved and deprotected by trifluoroacetic acid, then purified by preparative high pressure chromatography in inverse phase and lyophilized. Their purity (>95%) and their identity were confirmed by analytic HPLC and by mass spectrometry. Peptide sequences: SynB3 (H-RRLSYSRRRF-NH2; 1394.8 Da, SEQ ID No. 7 in the list of sequences in the appendix), SynB4 (H-AWSFRVSYRGISYRRSR-NH₂; 2145.1 Da, SEQ ID No. 6 in the list of sequences in the appendix), Tal-HLH (H-QQNVNGAFAELRKLIPTHPPDKKLSKNEILRLAMKYINFLA-NH2, SEQ ID No. 1 in the list of sequences in the appendix).

2) Coupling of Inhibitors on Peptide Vectors

Activation of the vectors: The peptide vectors SynB3 and SynB4 were treated by SPDP (N-Succinimidyl 3-(2-Pyridylthio)propionate) in Dimethylformamide (DMF) in the presence of DIEA (Diisopropylethylamine). The resulting peptides (2-Pyridylthio)propionyl-SynB3 (1591.8 Da) and (2-Pyridylthio)propionyl-SynB4 (2342.1 Da) were purified by HPLC and lyophilized.

Preparation of the conjugates: Each activated peptide vector (2-Pyridylthio)propionyl-SynBx (1 eq) was solubilized with C-TalHLH (1 eq) in DMF, in the presence of DIEA so as to form a disulphide bridge between the lateral chain of the Cystein of C-TalHLH (Tal-HLH compound synthesized with an additional cysteine) and 3-Mercapto-propionate carried by the vector. The resulting conjugates (Tal-HLH-SynB3; 6303 Da and Tal-HLH-SynB4; 7052.6 Da) were precipitated by the addition of ether, then purified by HPLC and lyophilized. The quality control by analytic HPLC at 220 nm and by MALDI-TOF mass spectrometry confirmed the molar masses and determined a purity exceeding 96%.

II—Compounds Tested

The compounds tested are presented in Table I below.

TABLE 1

| Compound | Name | Sequence |
|---|---|---|
| 1 | Tal-HLH | 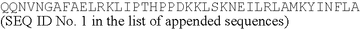 |
| 2 | Tal-hel 1rb | 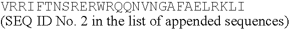 |
| 3 | Tal-hel 2b | 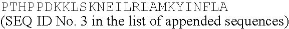 |
| 4 | TalHLH- SynB4 | 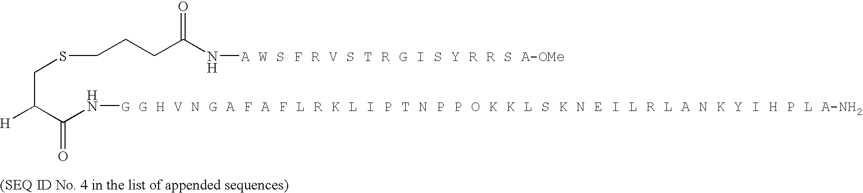 |
| 5 | TalHLH- SynB3 | 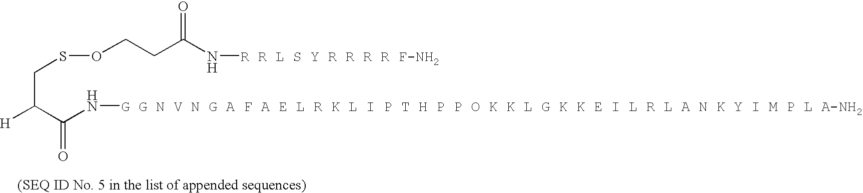 |
| 6 | SynB4 | 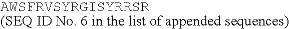 |
| 7 | SynB3 | 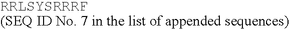 |

1) Translation/Immunoprecipitation Test

The two proteins TAL-1 and E47 are co-translated in vitro using plasmids enabling the transcription and translation of sequences coding in the system TNT-T7-coupled Reticulocyte Lysate, according to the conditions proposed by the supplier (Promega). The translation is carried out in the presence of methionine S³⁵, with or without the addition of peptides dissolved in a 2M solution of urea. An aliquot part (1 μl) of each translation is controlled by electrophoresis and autoradiography of the gel. The addition of up to 0.7 μg of peptide in the reticulocyte lysate (final volume of 25 μl) does not affect the efficacy of the translation of the two proteins.

After incubation for 30 min. at 37° C., the translation products are immunoprecipitated by a mixture of anti-TAL-1 monoclonal antibodies (BTL 73+2TL 136; (Pulford et al., Blood 85; 675 (1995))) and then analysed by electrophoresis and autoradiography of the gel. The TAL-1-E47 interaction is quantified for each point by calculating the ratio E47/TAL-1 obtained after the autoradiography scan. The intensity of the band corresponding to TAL-1 is used here for standardization in all the wells. The interaction in the absence of peptide is arbitrarily established at 100%.

2) Cytotoxicity Test

The haematopoietic cells K562 were commercially obtained from ATCC. The cells are seeded with about 10⁴ cells per well, 24 h before the addition of the products. They are then at a confluence of 60-80% on the day of the experiment. The cells are maintained in culture at 37° C. in an atmosphere at 95% humidity and 5% CO₂ in an OtimMem® medium.

The cells are incubated with increasing concentrations of the compounds for 48 hours. At the end of the culture time, the MTT (3-(4,5-dimethylthiazole-2-yl)-2,5-diphenyltetrazolium bromide) is added to the wells and the culture plates are then incubated for 4 hours in the oven. The resulting crystalline deposit of formazan is then dissolved with the addition of 200 μl of DMF/SDS. The optical density (OD) is measured at 550 nm (reference 630 nm) using a microplate reader.

The graphic representation of the percentages of OD of the wells treated as a function of the product concentration is used to determine the LD₅₀. This corresponds to the concentration of the product inhibiting 50% of the growth.

2) Survival Test of the Endothelial Cells

The human endothelial cells derived from the vein of the umbilical cord, HUVEC, (Clonetics) are cultivated on dishes coated with gelatine in the complete EMB-2 medium (Clonetics) supplemented with 10% decomplemented foetal calf serum. The cells are used between passages 3 and 5. The ECV 304 cells (obtained from ATCC) are cultivated in DMEM with 10% decomplemented foetal calf serum.

The cells are removed with trypsine and suspended in a medium without serum (OptiMEM, Invitrogen). About 8 10³ cells in 200 μl are incubated for 20 min. in the presence of different concentrations of peptides, centrifuged, then seeded in complete EBM2 medium in wells (48-wells plate) coated with collagen I. After 24 hours in the oven, the cell survival is estimated with an MTT test, carried out according to the supplier's (Sigma) recommendations.

III—Results

1) Effect of Inhibiting Peptides on the In Vitro Interaction of TAL-1/E47

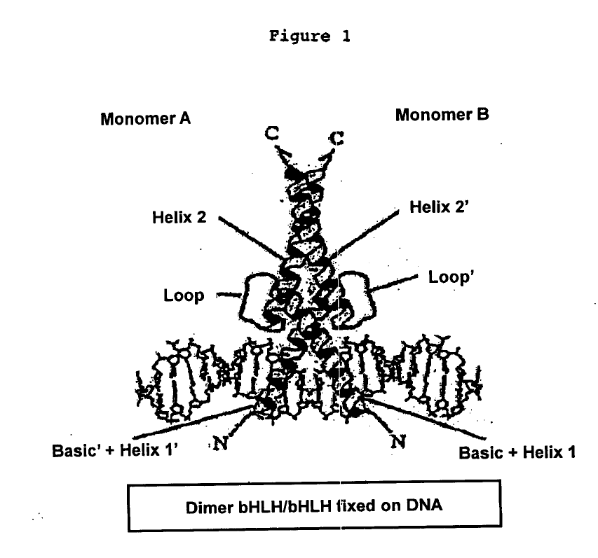

We chose to test whether the compounds described in Table 1 (Compounds 1, 2 and 3) could affect the formation of hetero dimers TAL-1-E47, mediated by the HLH domain of the two proteins. Compound 1 contains the entire HLH domain of TAL-1 or helix 1-loop-helix 2; compound 2 comprises the basic region and helix 1: compound 3 contains the loop and helix 2 (see FIG. 1).

We used a co-immunoprecipitation test for TAL-1 and E47 translated in vitro by antibodies directed against one of the proteins.

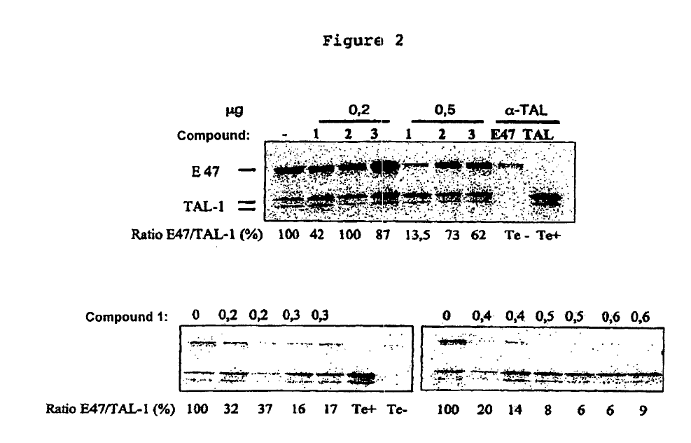

Our experiments demonstrate that entire compound Tal-HLH (compound 1) effectively inhibits the interaction of TAL-1 with E47 in a dose-dependant manner (50 to 60% inhibition with 0.2 μg; 90 to 100% with 0.5 μg) (FIGS. 2A and B). The other two inhibiting peptides (compounds 2 and 3) have a lower inhibiting power and do not exceed 50% with the highest concentrations (FIG. 2A). We therefore chose to use inhibiting peptide Tal-HLH (compound 1) for the rest of the experiments.

2) Effect of Inhibiting Peptides Coupled with Peptide Vectors on the In Vitro Interaction of TAL-1/E47

We wanted to determine whether the vectorization or coupling of inhibiting peptide Tal-HLH does not change its inhibiting effect on the in vitro interaction of TAL-1 with E47. The inhibitor was coupled with two peptide vectors (SynB3 and SynB4) via a disulphide bond (compounds 5 and 4, respectively). These two peptide vectors have the ability to increase the transport of molecules through the cell membranes. The results presented in fFIG. 3 confirm that the vectorization of inhibiting peptide Tal-HLH does not modify its inhibiting effect. It is interesting to note that the inhibitor coupled with vector SynB3 (compound 5) turned out to be more active than the Tal-HLH inhibitor (compound 1).

3) Effect of Inhibiting Peptides Coupled on Haematopoietic Cells

The inhibiting effect of vectorized inhibitor was first studied on a haematopoietic line (K562) whose survival requires the activity of TAL-1. The haematopoietic line T (H9) was used as a control since its growth is independent of TAL-1. These two leucemic lines can survive for several days in the absence of growth factors. This can be used to test the effect of peptides in a medium without serum.

As expected, a significant difference was not observed in the H9 cells (TAL-1 negative), whether the peptide Tal-HLH is vectorized or not (results not shown). However, Tal-HLH peptide had a higher cytotoxic effect on the K562 cells (requiring the action of TAL-1) when vectorized (Table 2). In fact, the LD50, corresponding to the concentration inhibiting 50% of the growth, is twice as low for the vectorized inhibitor (compound 4) than for the inhibitor alone (compound 1).

TABLE 2

| Compound | Name | LD 50 (μM) |
|---|---|---|
| Compound 1 | Tal-HLH | 11.5 |
| Compound 2 | TalHLH-SynB4 | 5.7 |
| Compound 3 | SynB4 | 12.3 |

These results are a first validation that vectorized peptide Tal-HLH (compound 4) is able to specifically inhibit the activity of TAL-1 in the cells.
4) Effect of Coupled Inhibiting Peptides on the Survival of Endothelial Cells

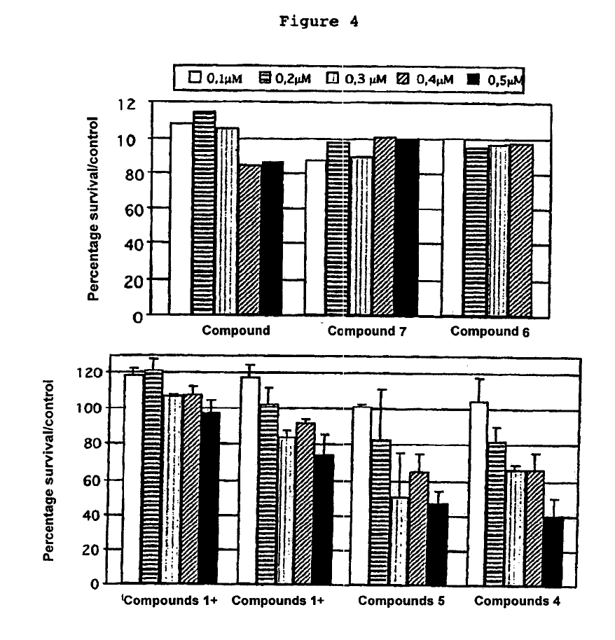

We also studied the effects of free and vectorized inhibitor on the survival of endothelial cells. FIG. 4 shows that compound 1 did not have an effect on the survival of HUVEC cells. However, once coupled (compounds 4 and 5), it specifically affects the survival of the HUVECs cells in a dose-dependant manner. The coupling of the product by a peptide vector was important to produce an effect on survival since the simultaneous addition of inhibitor and non-coupled peptide vector did not have any effect. In the ECV 304 cells used as a negative control, the compounds induced a non specific cell toxicity whether or not coupled (results not shown).

These experiments demonstrate the specific effect of the TAL-1 inhibitor when it penetrates in the endothelial cells with the peptide vector.

5) Effect of Peptides on the In Vitro Tubulogenesis of Human Endothelial Cells (Collagen Gels in 3-D)

The HUVEC endothelial cells seeded within a concentrated (1 mg/ml) collagen gel I stop proliferating and, in the presence of activation medium (MDCB131, 1% foetal calf serum, VEGF 2 ng/ml, bFGF 20 ng/ml and PMA 80 nM), they organize quickly in cell cords to form a primitive network. In 24-48 hours, the aligned cells grow longer, merge together and progressively form pseudo tubules. We tested the effects of vectorized peptide HLH (compound 1) in this in vitro system of tubulogenesis of human endothelial cells derived from umbilical cord (HUVECs). Increasing concentrations of vectorized peptide (compound 5) or cargo alone (compound 1) were added both to the collagen solution containing the cells and the activation medium.

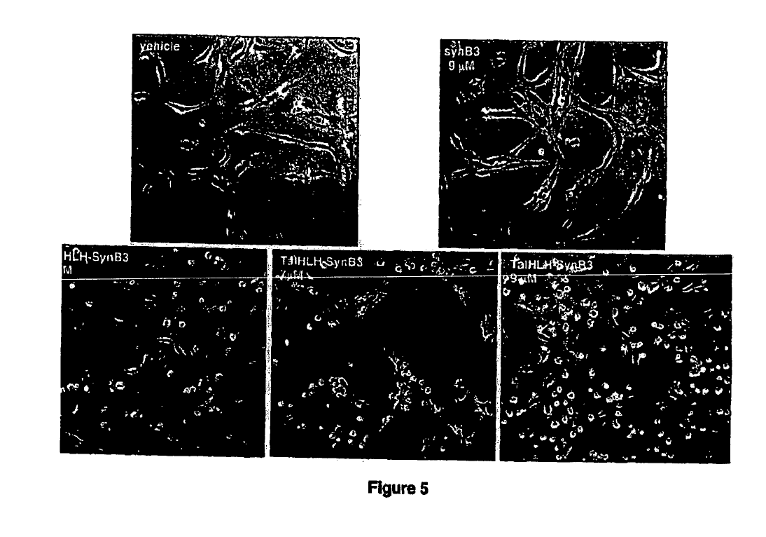

FIG. 5 illustrates one of these experiments: vectorized peptide HLH (compound 5) very strongly affects the tubulogenesis between 7 and 10 μM while the cargo alone (compound 1) does not have an effect on this range of concentrations with respect to the control cultures (5% DMSO).

6) Effect of Peptides on In Vivo Angiogenesis in the Mouse (Matrigel Plugs)

The Matrigel plug test consists of a sub-cutaneous injection in the mouse of the Matrigel, an extracellular matrix derived from a murine tumour, able to trigger neovascularization within the implant. In fact, the endothelial cells of the host, under the influence of the pro-angiogenetic factors contained in the Matrigel, migrate towards this “pseudo-tumour” and infiltrate in the Matrigel to organize a network of capillaries between 5 and 7 days.

In the experiments described above, two sub-cutaneous injections of Matrigel (500 μl) were carried out on both ends of the median line of the abdomen. Just before the injection, the mice were anaesthetized by inhalation of isoflurane (Forène®).

The Matrigel (BDBiosciences; batch 10403, 13 mg/ml) was complemented by bFGF (500 ng/ml), heparin (60 U/ml) and different peptides SynB3 (compound 7), SynB3-HLH (compound 5), SynB4 (compound 6), SynB4-HLH (compound 4) were added with a final concentration of 20 μM in 5% DMSO.

The mice were divided into 3 identical groups of six animals (males C57BL/6, 6 weeks old):
each mouse in the control group (mice 1 to 6) received two injections of Matrigel containing 5% DMSO on the left side and a control anti-angiogenic peptide on the right side,
each mouse in the “SynB3” group (mice 7 to 12) received two injections of Matrigel containing SynB3 on the left side and SynB3-HLH on the right side,

1-18. (canceled)

19. A peptide molecule that interferes with an HLH domain of TAL-1 comprising at least 10 successive amino acids from the HLH domain of TAL-1 of sequence:
(SEQ ID No. 1)
QQNVNGAFAELRKLIPTHPPDKKLSKNEILRLAMKYINFLA
or an equivalent sequence.

20. The peptide molecule according to claim 19, associated with a vector.

21. The peptide molecule according to claim 20, wherein the vector is selected from the group consisting of:
a linear peptide derived from protegrins or tachyplesins,
a linear peptide comprising a domain of transduction of TAT protein of HIV-1 domains of transduction derived from the third helix of Antennapdia,
liposome particles, and
polyethylene glycol (PEG) polymers.

22. The peptide molecule according to claim 20, wherein the vector is a linear peptide derived from Protegrins, complying with formula (I):
BX(X or B)BXXXXBBBXXXXXXB (I)
or a linear peptide derivative of Tachyplesin, complying with formula (II):
(B or X)XXXBXXXBXXXXBBXB (II),
in which:
identical or different B groups, representing an amino acid residue whose lateral chain bears an alkaline group, and
identical or different X groups, representing an aliphatic or aromatic amino acid residue or a fragment thereof consisting of a sequence of at least 5 successive amino acids of formulae (I) or (II).

23. The peptide molecule according to claim 20, wherein a bond between the molecule and the vector is selected from the group consisting of a covalent bond, a hydrophobic bond, an ionic bond, a cleavable bond or a non-cleavable bond in the physiological media or inside the cells.

24. The peptide molecule according to claim 23, wherein the bond may be either direct or indirect by a linker and carried out by a functional group that is naturally present or introduced either on the vector or on the inhibitor, or on both.

25. The peptide molecule according to claim 19, comprising:
compound 4:

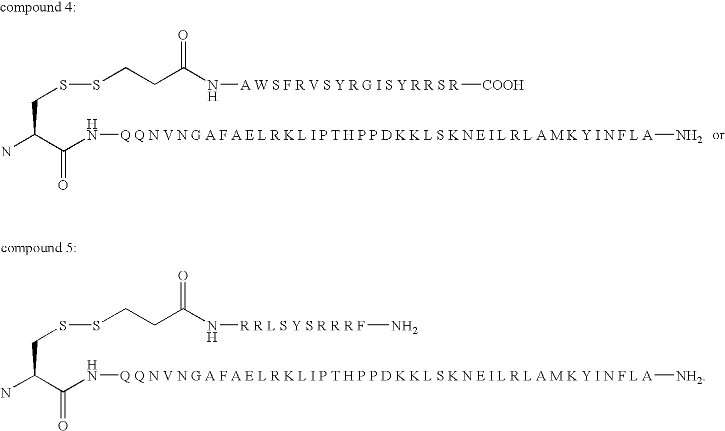

26. The peptide molecule according to claim 19, comprising at least 15 successive acids from SEQ ID No. 1.

27. A pharmaceutical composition comprising at least one peptide molecule as an active ingredient according to claim 19, and an acceptable vehicle.

28. The pharmaceutical composition according to claim 27, adapted for parenteral, oral, rectal, nasal, transdermal, pulmonary or central administration.

29. A method of preventing and/or treating diseases related to an angiogenesis, comprising administering a therapeutically effective amount of the pharmaceutical composition according to claim 27.

30. The method according to claim 29, wherein the diseases are selected from the group consisting of cancer, arteriosclerosis and diabetes.

31. A peptide molecule comprising a sequence selected from the group consisting of:
(SEQ ID No. 1)
QQNVNGAFAELRKLIPTHPPDKKLSKNEILRLAMKYINFLA,
(SEQ ID No. 2)
VRRIFTNSRERWRQQNVNGAFAELRKLI,
(SEQ ID No. 3)
PTHPPDKKLSKNEILRLAMKYINFLA
and a sequence equivalent to the sequences.

32. A pharmaceutical composition comprising at least one peptide molecule as an active ingredient according to claim 20, and an acceptable vehicle.

33. A method of preventing and/or treating diseases related to an angiogenesis, comprising administering a therapeutically effective amount of the pharmaceutical composition according to claim 32.

34. The method according to claim 33, wherein the diseases are selected from the group consisting of cancer, arteriosclerosis and diabetes.

35. A method of identifying a biologically active compound likely to be used in prevention and/or treatment of diseases related to angiogenesis, comprising detecting inhibition of interaction between an HLH domain of TAL-1 and its partner E47 in the presence of the compound.

36. The method according to claim 35, further comprising:
a) contacting protein TAL-1 or a fragment of the protein comprising the HLH domain, transcription factor E47 or a fragment of the factor comprising the HLH domain and the biologically active compound,
b) immunoprecipitating either 1) the HLH protein HLH or the fragment of the protein comprising the HLH domain or 2) transcription factor E47 or the fragment of the factor comprising the HLH domain,
c) if, at stage (b), the TAL-1 protein or the fragment of the protein comprising the HLH domain is immunoprecipitated, detecting in the immunoprecipitate obtained in stage (b), presence of a transcription factor E47 or the fragment of the factor comprising the HLH domain,
d) if, in stage (b), transcription factor E47 or the fragment of the factor comprising the HLH domain is immunoprecipitated, detecting in the immunoprecipitate obtained in stage (b), presence of protein TAL-1 or the fragment of the protein comprising the HLH domain, wherein, when the transcription factor E47 or the fragment of the factor comprising the HLH domain is not present in stage (c) or when the protein TAL-1 or the fragment of the protein comprising the HLH domain is not present in stage (d), the compound is an agent likely to be used in the prevention and/or treatment of diseases related to angiogenesis.

37. The method according to claim 35, characterized in that the method comprises the following stages:
(a′) contacting TAL-1 protein or a fragment of the protein comprising the HLH domain, transcription factor E47 or a fragment of this factor comprising the domain that interacts with TAL-1 and the biologically active compound,
(b′) causing the mixture obtained in stage (a′) to migrate on a non-denaturant polyacrylamide gel,
(c′) visualizing absence or presence of the TAL-1 complex or the fragment of the protein comprising the HLH domain and E47 or the fragment of the factor comprising the domain that interacts with TAL-1.
